(19) United States
(12) Patent Application Publication
(10) Pub. No.: US 2021/0046136 A1
(43) Pub. Date: Feb. 18, 2021

(54) METHOD FOR IMPROVING SLEEP ONSET AND SLEEP MAINTENANCE, METHOD FOR REDUCING STRESS AND IMPROVING RELAXATION, METHOD FOR IMPROVING PERFORMANCE AND CONCENTRATION, AND METHOD FOR IMPROVING RESTING EFFECT AND PROMOTING RECOVERY FROM FATIGUE

(71) Applicant: TOKIWA PHYTOCHEMICAL CO., LTD., Sakura-shi, Chiba (JP)

(72) Inventor: Jinwei YANG, Sakura-shi (JP)

(21) Appl. No.: 17/085,148

(22) Filed: Oct. 30, 2020

Related U.S. Application Data
(63) Continuation of application No. 15/938,618, filed on Mar. 28, 2018, now abandoned.

(30) Foreign Application Priority Data
Oct. 2, 2017 (JP) 2017-193127

(51) Int. Cl. A61K 36/24; A61K 31/7048; A61P 43/00

(52) U.S. Cl. CPC A61K 36/24; A61P 43/00; A61K 31/7048

(57) ABSTRACT

A method that promotes sleep onset and sleep maintenance, reduces stress, and improves a relaxation effect, performance, concentration, a resting effect and an effect of recovery from fatigue. The method includes administering to a subject Apocynum venetum leaf extract.

### CROSS REFERENCE TO RELATED APPLICATIONS

This is a continuation of U.S. patent application Ser. No. 15/938,618, filed Mar. 28, 2018, the disclosure of which is hereby incorporated by reference in its entirety.

### TECHNICAL FIELD

The present invention relates to a composition that can promote sleep onset and sleep maintenance, reduce stress, improve a relaxation effect, performance, concentration, and a resting effect, and promote recovery from fatigue.

### BACKGROUND ART

Sleep in modern people is reduced in quality and quantity due to various external environmental factors. In a study conducted by the Ministry of Health, Labor and Welfare in 2011, in practice, more than 50% of both males and females were susceptible to sleep problems (Non-Patent Literature 1). Markets for health foods and beddings for improving the quality of sleep are expanding, and particularly, products that can improve the experience of a sleep improvement effect are desirable.

In addition, people are exposed to various types of stress such as work environments, family issues, and human relations, and health problems such as depression, insomnia, and suicide are social problems. In order for people to adjust to modern society and live healthy lives, stress should be reduced, and it is also desirable to increase resistance to stress, and to reduce stress and solve problems related to performance, concentration, and nervousness by taking a supplement that is easily available.

Here, Apocynum venetum (Apocynum venetum L.) belongs to the family Apocynaceae, and is a perennial herb that naturally grows in China and temperate regions of Europe and Asia. Apocynum venetum leaves are drunk as a tea in China and an effect of lowering blood pressure has been proved in Japan, and Apocynum venetum leaves are certified as food for specified health uses.

In recent years, a sleep improvement effect of Apocynum venetum leaves has been focused on. It has been reported that, in a clinical test in which an electroencephalograph was worn by a subject at night and sleep was evaluated using an electroencephalogram, Apocynumvenetum leaf extract had an effect of prolonging non-REM sleep which is deep sleep (Non-Patent Literature 2).

In addition, regarding stress reduction and a relaxation effect, it has been reported that, in animal tests, Apocynum venetum leaf extract had an antidepressant effect (Non-Patent Literature 3 and Patent Literature 1) and exhibited an anxiolytic action (Non-Patent Literature 4). Here, since Apocynum venetum extract acts as an agonist of a cerebral inhibitory neurotransmitter GABA receptor, when a 25 mg GABA and a 25 mg Apocynum venetum leaf extract were ingested at the same time each day in a clinical test, the stress indicator chromogranin A was reduced, but a relaxation effect was not obtained when a 25 mg Apocynum venetum leaf extract was ingested alone each day (Patent Literature 2).

### CITATION LIST

### Non-Patent Literature

Non-Patent Literature 1
The National Health and Nutrition Survey in Japan, 2011, Part 3 Survey on Lifestyle Habits
Non-Patent Literature 2
Yamatsu A, Yamashita Y, Maru I, Yang J, Tatsuzaki J, Kim M., “The Improvement of Sleep by Oral Intake of GABA and Apocynum venetum Leaf Extract”, J Nutr Sci Vitaminol (Tokyo), 61(2), 182-187(2015).
Non-Patent Literature 3
Butterweck V, Nishibe S, Sasaki T, Uchida M., “Antidepressant effects of apocynum venetum leaves in a forced swimming test”, Biol Pharm Bull., 24(7), 848-851 (2001).
Non-Patent Literature 4
Grundmann O, Nakajima J, Kamata K, Seo S, Butterweck V., “Kaempferol from the leaves of Apocynum venetum possesses anxiolytic activities in the elevated plus maze test in mice” Phytomedicine, 16, 295-302 (2009).

### Patent Literature

Patent Literature 1
Japanese Patent No. 4629933
Patent Literature 2
Japanese Unexamined Patent Application Publication No. 2011-93842

### SUMMARY OF INVENTION

### Technical Problem

Incidentally, regarding sleep improvement, consumers prefer products that give an excellent feeling of sleep improvement to those that have objective evaluation indicators. Among the reports studied so far, the Apocynum venetum leaf extract has been known to have an effect of prolonging time of non-REM sleep. However, shortening of a sleep onset time or an effect for an arousal rate were not confirmed and subjects' feelings of sleep improvement were weak.

In addition, due to insomnia and stress, productivity on the next day decreases, concentration decreases, work and operations are increasingly unlikely to be performed smoothly, and it is reported that there is an economic loss of 3.5 trillion yen per year, which causes societal problems. It is unknown whether Apocynum venetum extract has an effect on stress, daytime work performance, or concentration.

### Solution to Problem

In view of the above problem, the inventors conducted studies on the Apocynum venetum leaf extract, and designed a clinical test similar to daily sleep conditions except for the negative influence on sleep of wearing an electroencephalograph, and as a result, verified that a sleep improvement effect, and particularly, a feeling of sleep improvement that is most demanded by consumers, is obtained, and thus completed the present invention.

Here, it is confirmed in a new clinical test that the Apocynum venetum extract reduces stress and has a relaxing effect when an intake of the Apocynum venetum leaf extract is increased. Further, it is proved first that it has effects of improving daytime performance, concentration, quality of rest, and recovery from fatigue that have not been reported so far.

That is, a composition for improving sleep onset and sleep maintenance according to an aspect of the present invention includes an Apocynum venetum leaf extract as an active ingredient.

In addition, a composition for reducing stress and improving relaxation according to another aspect of the present invention includes an Apocynum venetum leaf extract as an active ingredient.

In addition, a composition for improving performance and concentration according to still another aspect of the present invention includes an Apocynum venetum leaf extract as an active ingredient.

In addition, a composition for improving a resting effect and promoting recovery from fatigue according to yet another aspect of the present invention includes an Apocynum venetum leaf extract as an active ingredient.

### Advantageous Effects of Invention

As above, according to the present invention, it is possible to provide a composition that can promote sleep onset and sleep maintenance, reduce stress, improve a relaxation effect, performance, concentration, and a resting effect, and promote recovery from fatigue.

### DESCRIPTION OF EMBODIMENTS

Embodiments of the present invention will be described below in detail. However, the present invention can be embodied in many different forms, and is not limited to only examples described in the following embodiments and examples.

An embodiment of the present invention is a composition that can promote sleep onset and sleep maintenance, reduce stress, improve a relaxation effect, performance, concentration, and a resting effect, and promote recovery from fatigue.

The Apocynum venetum leaf extract of the embodiment of the present invention is an extract that is obtained by extracting leaves of Apocynum venetum leaf (binomial name: Apocynum venetum L.) in a solvent including at least one of water, ethanol, aqueous ethanol, and an organic solvent and performing condensation, an extract that is obtained by adsorbing the obtained extract to at least one of acrylic, styrene and methacrylic and aromatic synthetic adsorbents, and condensating fractions eluted with aqueous ethanol up to a concentration of 10 to 95%, and an extract obtained by drying the extract.

In addition, the present composition is in the form of food, medicine, or cosmetics. In particular, in the form of food, it is in the form of a functional display food or health food. Furthermore, in the form of food, it is in the form of not only a solid substance but also beverage.

In addition, when the present composition is in the form of food, the form of desserts, for example, drinks, candies, jellies, and gummies, can be exemplified. When the present composition is in the form of a health food or food with function claims, the form of, for example, a tablet, a hard capsule, a soft capsule, granules, and a drink can be exemplified.

In addition, when the present composition is a pharmaceutical product, the form of the pharmaceutical product can include a tablet, a capsule, a pill, a liquid, and an emulsion. An administration method is not particularly limited. However, an oral administration form is desirable. In addition, various carriers can be added within a pharmaceutically acceptable range. Examples of the carriers can include an excipient, a colorant, a sweetener, and a suspending agent.

In addition, a content of the Apocynum venetum extract in the present composition can be appropriately adjusted according to an assumed intake.

In addition, in order to promote sleep onset and sleep maintenance, reduce stress, improve a relaxation effect, performance, concentration, quality of rest, and an effect of recovery from fatigue according to the present composition, the Apocynum venetum leaf extract is preferably contained at 30 mg or more and 150 mg or less, and more preferably at 35 mg or more and 125 mg or less, and when it is contained at 40 to 100 mg/day, such effects can be positively obtained.

In addition, the Apocynum venetum leaf extract in the present composition contains hyperoside and isoquercitrin which are flavonoid compounds as an active ingredient. A total amount thereof is preferably 4 weight % or more, and more preferably is in a range of 4 weight % or more and 10 weight % or less so that the effects of the present composition are more favorably exhibited.

Incidentally, the present composition promotes sleep onset and sleep maintenance, reduces stress, and improves a relaxation effect, performance, concentration, a resting effect, and an effect of recovery from fatigue. Here, a range in which such effects can be obtained can also be clearly understood from description in the following example, which is the same as above.

As above, according to the present invention, it is possible to provide a composition that promotes sleep onset and sleep maintenance, reduces stress, and improves a relaxation effect, performance, concentration, a resting effect, and an effect of recovery from fatigue.

### EXAMPLES

Here, the composition according to the above embodiment was actually prepared and effects thereof were checked. Details will be described below.

### (1) Preparation of Apocynum Venetum Leaf Extract

Dried Apocynum venetum leaves were pulverized by a laboratory mixer, 60% ethanol (6 L) was added to the pulverized product (1 kg), and the mixture was heated and circulated for 2 hours, and then filtered to obtain an extract solution. The extraction residue was heated and circulated again in 60% ethanol (6 L) for 2 hours and then filtered to obtain an extract solution. The first extract solution and the second extract solution were combined, condensated under a reduce pressure at 60 degrees, condensated to about ⅕ volume, and then suspended in water in an amount of two times thereof. Then, a pH of the mixture was adjusted to 3 using citric acid, and stirring was performed overnight. Diatomaceous earth was added to form a body feed, the solution was poured onto a filter paper with the diatomaceous earth spread thereon, and insoluble substances were removed by filtration. The obtained filtrate was passed through a synthetic adsorption resin HP20 (1 L) (commercially available from Mitsubishi Chemical Corporation), an active ingredient was adsorbed, and then washing with water (2 L) was performed to remove saccharide and the like. Then, desorption was performed in 70% ethanol (2 L), fractions containing an active ingredient were collected, condensated under a reduced pressure at 50 degrees, and condensated to about 1/10 volume, spray drying was then performed, and thereby a solid substance (95 g) was obtained.

Here, when measurement of the solid substance obtained above was performed by liquid chromatography, it was confirmed that hyperoside was contained at 2 to 5 weight %, and isoquercitrin was contained at 2 to 5 weight %, with a total amount of 4 to 10 weight %.

### (2) Clinical Test

In a clinical test, healthy Japanese adult males and females participated as subjects, and tablets containing the Apocynum venetum leaf extract (25 mg per tablet) were used. Here, as a placebo, a food additive coloring formulation having the same appearance without the Apocynum venetum leaf extract was used as a test food.

TABLE 1

| | | name of raw materials | | |
|---|---|---|---|---|
| name | shape | (per tablet) | blending amount | |
| a Apocynum | tablet | Apocynum venetum leaf | 25.00 | mg |
| venetum leaf | | extract | | |
| extract | | starch decomposition product | 84.00 | mg |
| tablet | | crystalline cellulose | 59.50 | mg |
| | | lactose | 22.70 | mg |
| | | edible fat and oil | 6.30 | mg |
| | | glycyrrhiza uralensis extract | 2.50 | mg |
| | | gum guaiac | a very small mount | |
| | | shellac | a very small mount | |
| | | glyceryl fatty acid ester | a very small mount | |
| | | carnauba wax | a very small mount | |
| placebo | tablet | starch decomposition product | 84.00 | mg |
| tablet | | crystalline cellulose | 59.50 | mg |
| | | caramel pigment | 25.00 | mg |
| | | lactose | 22.70 | mg |
| | | edible fat and oil | 6.30 | mg |
| | | glycyrrhiza uralensis extract | 2.50 | mg |

| | |
|---|---|
| gum guaiac | a very small mount |
| shellac | a very small mount |
| glyceryl fatty acid ester | a very small mount |
| carnauba wax | a very small mount |

A randomized, placebo-controlled, double blind, crossover comparative study was performed with 17 subjects. Until days 1 to 7, an intervention group ingested the Apocynum venetum leaf extract (50 mg/day) 30 minutes to 1 hour before going to bed, and a placebo group ingested the same number of the placebo tablets. On the 8th day, they ingested the tablets 15 minutes to 30 minutes before the questionnaire started before an Uchida-Kraepelin psychodiagnostic test.

Immediately after ingestion for 7 days, a feeling of sleep improvement was evaluated using an OSA sleep questionnaire MA version. For scores of question items, scale values for the items were referred to. The score was obtained by an average value of the question items. A higher score indicates a favorable sleep state.

Here, on the 8th day, the Uchida-Kraepelin psychodiagnostic test was performed, and calculation was performed within a determined time. An amount of calculation, a calculation accuracy rate, and a perturbation rate were measured to evaluate improvement in performance and concentration. In addition, the questionnaire was performed before and after the Uchida-Kraepelin psychodiagnostic test, and improvement in stress and a relaxation effect were evaluated. Here, the Uchida-Kraepelin psychodiagnostic test was performed for 15 minutes each in the first half and the second half in a divided manner, with a 5-minute rest therebetween, work efficiencies in the first half and the second half were compared, and efficiency of rest and recovery of fatigue were evaluated.

### Results of Clinical Test

### (3) Results of OSA Sleep Questionnaire

It was confirmed that the intervention group had a significantly higher factor II for evaluating sleep onset and sleep maintenance compared to the placebo group (placebo group: 1.9±3.6; intervention group 7.5±7.8, p=0.042)

### (4) Uchida-Kraepelin Psychodiagnostic Test

### Questionnaire

In evaluation items “nervous,” “restless,” and “irritated” related to improvement in stress and a relaxing effect, the intervention group had significantly lower scores than the placebo group.

TABLE 2
variation (after ingesting the tablet − before ingesting the tablet)

| item | intervention group | placebo group | p value |
|---|---|---|---|
| nervous | −0.5 ± 0.7 | 0.2 ± 0.9 | 0.015 |
| restless | −0.5 ± 0.9 | 0.3 ± 1.2 | 0.038 |
| irritated | −0.6 ± 1.1 | 0.1 ± 0.7 | 0.038 |

### (5) Uchida-Kraepelin Psychodiagnostic Test

The item “perturbation rate” related to performance and concentration was for evaluating the variation in work. A lower score indicates a smaller variation in work, higher concentration, and improved performance. As shown in the following table, the intervention group had a significantly lower perturbation rate than the placebo group.

TABLE 3
variation (after ingesting the tablet − before ingesting the tablet)

| item | intervention group | placebo group | p value |
|---|---|---|---|
| perturbation rate | −0.19 ± 0.23 | 0.09 ± 0.46 | 0.043 |

### (6) Uchida-Kraepelin Psychodiagnostic Test

In the evaluation item related to a resting effect and recovery from fatigue, it is significantly improved more in the intervention group than in the placebo group

TABLE 4
variation (after ingesting the tablet − before ingesting the tablet)

| item | intervention group | placebo group | p value |
|---|---|---|---|
| rest effect rate | 0.03 ± 0.09 | −0.05 ± 0.08 | 0.029 |
| rest elongation rate | 0.03 ± 0.09 | −0.05 ± 0.08 | 0.029 |

As shown above, in the above human test, it was confirmed that the composition promoted sleep onset and sleep maintenance, reduced stress, and improved a relaxation effect, performance, concentration, a resting effect, and an effect of recovery from fatigue.

1. A method for improving sleep onset and sleep maintenance comprising a step of:
administering to a subject a pharmacologically effective amount of Apocynum venetum leaf extract.

2. The method for improving sleep onset and sleep maintenance according to claim 1,
wherein the Apocynum venetum leaf extract contains 4 to 10% hyperoside and isoquercitrin which are flavonoid compounds.

3. The method for improving sleep onset and sleep maintenance according to claim 1,
wherein an intake of the Apocynum venetum leaf extract per day is in a range of 40 mg or more and 100 mg or less.

4. A method for reducing stress and improving relaxation comprising a step of:
administering to a subject a pharmacologically effective amount of Apocynum venetum leaf extract as an active ingredient.

5. The method for reducing stress and improving relaxation according to claim 4,
wherein the Apocynum venetum leaf extract contains 4 to 10% hyperoside and isoquercitrin which are flavonoid compounds.

6. The method for reducing stress and improving relaxation according to claim 4,
wherein an intake of the Apocynum venetum leaf extract per day is in a range of 40 mg or more and 100 mg or less.

7. A method for improving performance and concentration comprising a step of:
administering to a subject a pharmacologically effective amount of Apocynum venetum leaf extract as an active ingredient.

8. The method for improving performance and concentration according to claim 7,
wherein the Apocynum venetum leaf extract contains 4 to 10% hyperoside and isoquercitrin which are flavonoid compounds.

9. The method for improving performance and concentration according to claim 7,
wherein an intake of the Apocynum venetum leaf extract per day is in a range of 40 mg or more and 100 mg or less.

10. A method for improving a resting effect and promoting recovery from fatigue comprising a step of:
administering to a subject a pharmacologically effective amount of Apocynum venetum leaf extract as an active ingredient.

11. The method for improving a resting effect and promoting recovery from fatigue according to claim 10,
wherein the Apocynum venetum leaf extract contains 4 to 10% hyperoside and isoquercitrin which are flavonoid compounds.

12. The method for improving a resting effect and promoting recovery from fatigue according to claim 10,
wherein an intake of the Apocynum venetum leaf extract per day is in a range of 40 mg or more and 100 mg or less.

13. The method for improving sleep onset and sleep maintenance according to claim 1,
wherein the Apocynum venetum leaf extract is obtained by extracting leaves of Apocynim venetum leaf in a solvent including at least one of water, ethanol, and aqueous ethanol.

14. The method for reducing stress and improving relaxation according to claim 4,
wherein the Apocynum venetum leaf extract is obtained by extracting leaves of Apocynim venetum leaf in a solvent including at least one of water, ethanol, and aqueous ethanol.

15. The method for improving performance and concentration according to claim 7,
wherein the Apocynum venetum leaf extract is obtained by extracting leaves of Apocynim venetum leaf in a solvent including at least one of water, ethanol, and aqueous ethanol.

16. The method for improving a resting effect and promoting recovery from fatigue according to claim 10,
wherein the Apocynum venetum leaf extract is obtained by extracting leaves of Apocynim venetum leaf in a solvent including at least one of water, ethanol, and aqueous ethanol.

17. The method for improving sleep onset and sleep maintenance according to claim 1,
wherein the pharmacologically effective amount of Apocynum venetum leaf extract is conveyed by a pharmaceutically acceptable carrier.

18. The method for reducing stress and improving relaxation according to claim 4,
wherein the pharmacologically effective amount of Apocynum venetum leaf extract is conveyed by a pharmaceutically acceptable carrier.

19. The method for improving performance and concentration according to claim 7,
wherein the pharmacologically effective amount of Apocynum venetum leaf extract is conveyed by a pharmaceutically acceptable carrier.

20. The method for improving a resting effect and promoting recovery from fatigue according to claim 10,
wherein the pharmacologically effective amount of Apocynum venetum leaf extract is conveyed by a pharmaceutically acceptable carrier.
